(19) United States
(12) Patent Application Publication
(10) Pub. No.: US 2004/0073030 A1
(43) Pub. Date: Apr. 15, 2004

(54) Process for the preparation of quinolone derivatives

(76) Inventors: Reddy Muddasani Pulla, Banjara Hills, Hyderabad (IN); Chowdary Nannapaneni Venkaiah, Banjara Hills, Hyderabad (IN)

Correspondence Address: MERCHANT & GOULD PC, P.O. BOX 2903, MINNEAPOLIS, MN, 55402-0903, US

(21) Appl. No.: 10/332,759

(22) PCT Filed: Jun. 19, 2001
(86) PCT No.: PCT/IN01/00042

(30) Foreign Application Priority Data
May 9, 2000 (IN) 360/MAS/2000

(51) Int. Cl. C 07D 4 3/02; C07D 215/20

(52) U.S. Cl. 544/363; 546/156

(57) ABSTRACT

This invention relates to an improved process for the preparation of quinolone drugs of the formula (I), wherein R=C₁-C₆ alkyl, C₃-C₆ cycloalklyl, aryl, substituted aryl, NR¹R²=diarylamino, arylalkylamino, C₁-C₆-dialkylamino, piprazinyl, N or C alkyl (C₁-C₆) substituted piperazinyl, morpholino, pyrrolidinyl, substituted pyrrolidinyl, aralkyl, substituted aralkyl etc. Some of the compounds falling within the formula (I) are ciprofloxacin, enrofloxin, pefloxacin, etc. These compounds are useful as antibacterial drugs. The process of the present invention for preparation of compound of formula (I) comprises in enhancing the reactivity of the displaceable halogen (X) in the compound of the formula (II) towards various amines of formula (III) wherein R=as defined for compound of formula (I), R³=COOR⁶ (R⁶=C₁-C₆ alkyl, aryl, aralkyl), nitrile a carboxamide (—CONR⁷R⁸, R⁷ and R⁸=C₁-C₆ alkyl, C₃-C₆ cycloalkyl, aralkyl), X=Cl, Br, F; NR¹R²=as defined above by introducing a nitro group ortho to the displaceable halo group and subsequently removing the nitro group in a conventional manner. The process of the present invention enhances the yield of the compound of the formula (I) and also improves the quality of the prepared compound.

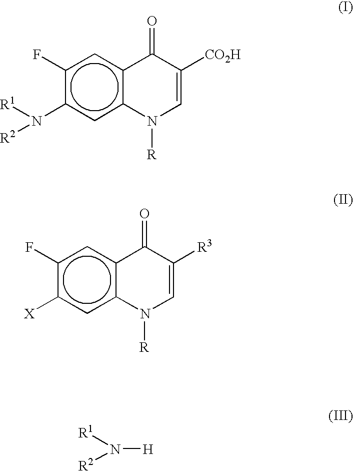

The present invention relates to an improved process for the preparation of quinolone derivatives. The present invention particularly relates to an improved process for the preparation of quinolone derivative of the general formula I.

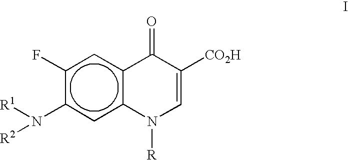

wherein R=C₁-C₆ alkyl C₃-C₆ cycloalkyl, aryl, substituted aryl, NR¹R²=diarylamino, arylalkylamino, C₁-C₆-dialkylamino, piprazinyl, N or C alkyl (C₁-C₆) substituted piperazinyl, morpholino, pyrrolidinyl, substituted pyrrolidinyl, aralkyl, substituted aralkyl etc.

The compounds of the general formula I are normally prepared by reacting the appropriate haloquinolone of the formula II

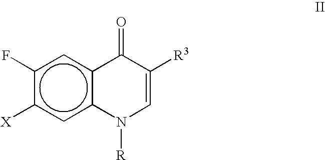

wherein R=as defined for compound of formula—I, R³-COOR⁶ (R⁶=C₁-C₆ alkyl, aryl, aralkyl), nitrile, a carboxamide (—CONR⁷R⁸, R⁷ and R⁸=C₁-C₆ alkyl, C₃-C₆ cycloalkyl, aralkyl), X=Cl, F

with an appropriate amine of the formula III

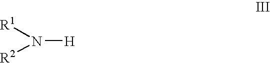

wherein NR¹R²=diarylamino, arylalkylamino, C₁-C₆-dialkylamino, piperazinyl, N or C alkyl (C₁-C₆) substituted piperazinyl, morpholino, pyrrolidinyl, substituted pyrrolidinyl, aralkyl, substituted aralkyl, etc.

in a polar protic or aprotic solvent at a high temperature for a prolonged period (U.S. Pat. Nos. 4,146,719 and 4,670,444). The above said reaction is illustrated as shown in scheme-1 below.

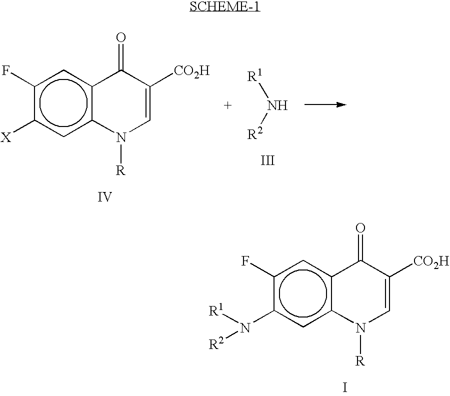

wherein R, R¹ and R² are as defined above.

### SCHEME-1

For example, Ciprofloxacin(I, R=cyclopropyl, NR¹R²=piperazinyl) and enrofloxacin (I, R=cyclopropyl, NR¹R²=4-ethyl- piperazinyl) are prepared in 70-75% yield by reacting a compound of formula—IV (R=cyclopropyl, X=Cl) with 4-5 equivalents of a compound of formula—III (NR¹R²=piperazine for ciprofloxacin and N-ethyl piperazine for enrofloxacin) in pyridine medium for 6- 8 hrs. at 120° C. (Grohe, et al, Liebigs Ann. Chem. 1087, 1, 29-37).

Similarly, norfloxacin (I, R=ethyl, NR¹R²=piperazinyl ) and pefloxacin (R=ethyl, NR¹R²=4-methyl- piperazinyl) are prepared in 66-68% yield by reacting a compound of formula—IV (R=ethyl, X=Cl) with 4-5 equivalents of a compound of formula—III (NR¹R²=piperazine for norfloxacin and N-methyl-piperazine for pefloxacin) with no solvent at 135-140° C. for 5 hrs. (Koga,et al, J. Med. Chem. 1980, 23 1358-1363).

The above said process has the following disadvantages:

1. Selectivity for the halogen displacement it only 90 to 95%. The remaining material is a fluorine displaced derivative.

2. The process requires more than 3 moles of the amine per mole of quinolonic acid.

3. The reaction requires very high temperature (more than 120° C.) and prolonged reaction time (4-8 hours).

4. Removal of impurity is a tedious process and recovery of the final product is not 100%.

5. The yield of the final product is normally less than 70%.

Another conventional process disclosed in PCT International application WO 88 07,993 involves the enhancement of halogen activity by chelating the β-keto acid part of quinolonic acid of formula IV with a boron derivative B(OAc)₃ of the formula (V). This reaction is shown in the Scheme-2 given below.

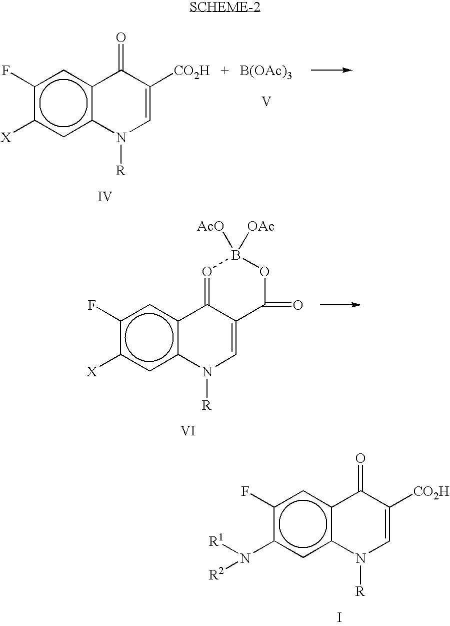

where in X, R, R¹ and R² are as defined above

For example, a compound of formula—IV (R=cyclopropyl, X=Cl) is reacted with boric acid triacetate (V) in acetic acid medium to get a compound of formula—VI (R=cyclopropyl, X=Cl) in almost quantitative yield. This compound is reacted with 3-4 moles of a compound of formula—III (NR¹R²=piperazine) in DMSO solvent medium for 10 mins. at 110-120° C. to get ciprofloxacin (I, R=cyclopropyl, NR¹R²=piperazinyl) in 95% yield.

This process has the following disadvantages:

1. The process requires high temperature (>100° C.)

2. The reaction time is too short (10 minutes) to perform it on a commercial scale.

3. The reaction works with only polar aprotic solvents like DMSO.

4. The reaction requires more than 3 moles of amine per mole of quinolonic acid.

5. Though the process claims to have more than 90% yield actually it never exceeds 75%.

Since the quinolone drugs like ciprofloxacin, enrofloxacin, etc falling within the general formula I given above are very useful broad spectrum antibacterial drugs there is a need to enhance the yield as well as the purity of the said compounds prepared. Accordingly there is an urgent need for developing an improved process for the preparation of the said compounds resulting in higher yields and improved purity.

Therefore the main objective of the present invention is to provide an improved process for the preparation of compounds of the general formula I defined above having improved yield (90-95%) as well as improved purity (≧99%).

Another objective of the present invention is to provide an improved process for the preparation of compounds of the general formula I as defined above by enhancing the reactivity of the halogen (X) in the formula IV given above towards various amines thereby reducing the formation of the impurity of the formula VII,

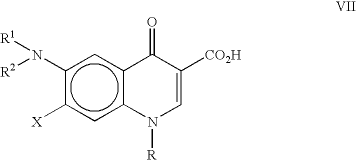

wherein X,R, R¹, R² are as defined above.

resulting in the increased purity of the final compound of the formula I.

Yet another objective of the present invention is to provide an improved process for the preparation of the compound of the formula I defined above which process involves a facile reaction on both of the halogens in the compound of the formula VIII.

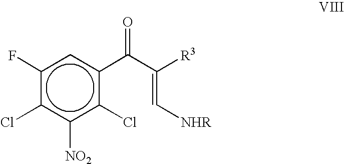

wherein R, R³ are as defined above.

The invention has been developed based on our finding that:

1. Both the chlorines present in the formula VIII undergo the reaction even at room temperature.

2. The low temperature reaction leads to higher yields (90-95%) and better purity (≧99%) of the compounds of the formula I.

3. The amount of the costly amine of the formula III (R¹R²NH) in the process requires only one equivalent.

4. The step of removing the impurity of formula VII is totally eliminated as no such impurity is formed.

Accordingly the present invention provides an improved process for the preparation of compound of the general formula I as defined above which comprises:

i. converting 2,4-dichloro-5-fluoroacetophenone to 2,4-dichloro-5-fluoro-3 nitrobenzoic acid by conventional methods.

ii. reacting the 2,4-dichloro-5-fluoro-3-nitrobenzoic acid with thionyl chloride/oxalyl chloride to get the corresponding benzoyl chloride.

iii. reacting the 2,4-dichloro-5-fluoro-3-nitrobenzoyl chloride with the compound of the formula IX

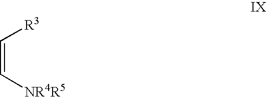

wherein R³=COOR⁶ (R⁶=C₁-C₆ alkyl, aryl, aralkyl), nitrile, and carboxamide (CONR⁷R⁸, R⁷ and R⁸=C₁-C₃ alkyl, cyclo alkyl, aralkyl), R⁴, R⁵=C₁-C₆ alkyl, aralkyl, cycloalkyl, (C₃-C₆) together with the nitrogen atom to which they are bonded to give a compound of formula-X.

to yield the corresponding novel acrylate of the formula X.

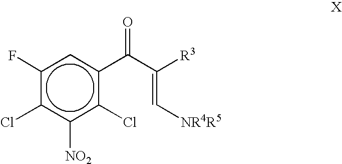

wherein R³, R⁴ and R⁵ are as defined above.

iv. reacting the novel compound of the formula X with a primary amine RNH₂ (of the formula XI) where R is as defined above to give a compound of the formula VIII.

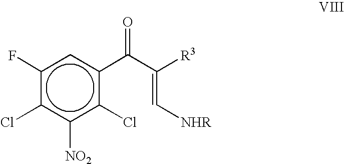

wherein R & R³ have the meanings given above.

v. reacting the compound of the formula VIII with a base in the presence of a polar aprotic or nonpolar solvent or their mixtures to give a new compound of the formula XII.

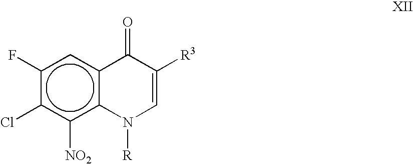

wherein R, R³ are as defined above.

vi. reacting the novel quinolone of the formula XII with an amine R¹NHR² (of the formula III), wherein R¹ and R² have the meanings given above to give the corresponding chloro displaced novel product of the formula XIII.

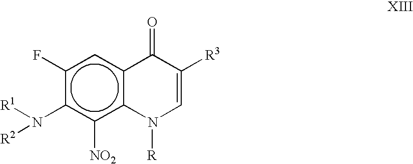

wherein R, R¹, R² & R³ have the meanings given above.

vii. subjecting the compound of the formula XIII to metal reduction by conventional methods to yield a novel compound of the formula XIV.

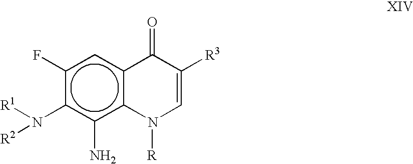

wherein R, R¹, R² & R³ have the meanings given above.

viii. deaminating the compound of the formula XIV via its diazonium salt and decomposing the salt by conventional methods to yield the compound of the formula I as defined above OR decomposing the diazonium salt of the compound of the formula XIV in the presence of copper (I) halide to give a novel halide derivative of the formula XV.

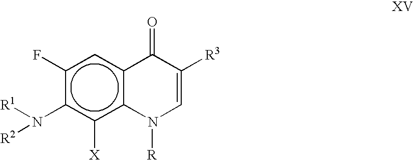

wherein R, R¹, R² & R³ have the meanings given above, and X=H, Cl, Br.

ix. dehalogenating the compound of the formula XV by conventional methods to yield the compound of the formula I as defined above.

In a preferred embodiment of the invention the step (i) above may be carried out following method contained in J. Heterocyclic Chemistry 1988, 25, 927. The nitro group present in this acid enhances the reactivity of both the Cl atoms in the subsequent steps.

The transamination in step (iv) may be done in routine solvents such as alcohols like methanol, ethanol, isopropanol, etc; aromatic solvents like benzene, toluene, etc. The reaction temperature may be in the range of 0 to 60° C. The preferred temperature being in the range of 0 to 25° C.

The base employed in step (v) may be MOR where M represents Na, K and R represents C₁ to C₄ atoms, carbonate salts like K₂CO₃, Na₂CO₃, metal hydride like NaH. The polar solvent employed may be selected from DMF, OMSO, DMAc etc and the nonpolar solvent can be benzene, toluene, xylene etc.

The step (vi) may be effected at a temperature in the range of 25 to 100° C., preferably in the range of 25 to 40° C. The medium which can be employed for the reaction may be a polar aprotic solvent such as DMF, DMSO, IPA, n-BuOH, t-BuOH etc. An aromatic solvent such as benzene, toluene, xylene, a halogenated solvent like CH₂Cl₂ CHCl₃, and acetonitrile. The reaction may also be conducted with one equivalent of amine and a base like Na₂CO₃, K₂CO₃, NaHCO₃, TEA etc.

The reducing agent which may be employed in step (vii) may be Raney nickel, Pd/C, FelAcOH, Sn/HCl etc. preferably Raney nickel or Pd/C (5 to 10%). The medium which can be employed may be selected from alcoholic solvent such as methanol, ethanol, isopropanol etc., esters like ethyl acetate. The preferred solvent may be methanol. The temperature employed in the reaction may be in the range of 20 to 100° C., preferably 20 to 40° C. The hydrogen pressure employed may be in the range of 10 to 60 psi preferably 20 to 40 psi.

The deamination step (viii) may be effected via the diazonium salt derived from a compound of formula XIV which can be decomposed in the presence of alcohol or hypophosphorous acid to give directly the compound of the formula I (XV, X=H) or it can be decomposed in the presence of copper (I) halide to give the halo derivative of the formula XV.

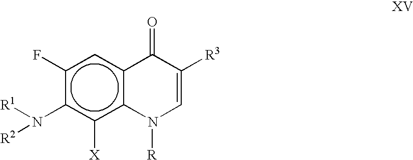

wherein X, R, R¹, R² & R³ have the meanings given above

The diazonium salt can be prepared in dil H₂SO₄ medium for its direct conversion to a compound of the formula I or in dil aq HX where X represents Cl or Br medium for its conversion to a compound of the formula XV. The decomposition temperature employed may be in the range of 5 to 80° C. preferably 15 to 30° C. for direct conversion to the compound of the formula I. For the conversion to the halo derivative of the formula XV, the temperature may be in the range of 20 to 40° C.

The halo derivative of the formula XV can be dehalogenated to give the oompound of the formula I by any conventional methods. The methods which can be employed may be selected from treating with Raney nickel or Pd/C under hydrogen atmosphere in the presence of acid scavenger to neutralise the liberalised acid. The catalyst which may be employed for the conversion may be preferably 5% Pd/C, The acid scavenger employed may be amines of the general formula R₃N where R represents C₁ to C₆ alkyl, or carbonate or bicarbonate salts of Na or K, preferably triethylamine. The temperature of reaction employed may be in the range of 20 to 100° C. preferably 20 to 40° C. The hydrogen pressure employed may be in the range of 10 to 60 psi preferably 20 to 40 psi. The solvent medium which can be employed may be selected from methanol, ethanol, isopropanol etc.

The protecting group present on NH group of NR¹R² may be deprotected by acid or base hydrolysis and also R³ (if it is an ester, amide or nitrile) can also be hydrolysed in one operation. The acid employed may be sulphuric acid in combination with water and/or acetic acid. The base employed may be a strong base like NaOH or KOH. The preferred hydrolysis for piperazine type amine with an acyl protection and R³ being an alkyl ester would be dilute sodium hydroxide (2 to 10% or NaOH) at a temperature in the range of 20 to 100° C., preferably at 40 to 60° C.

The invention is described in detail in the Example given below which is provided to illustrate the invention only and therefore it should not be construed to limit the scope of the invention.

### EXAMPLE1

(i) Preparation of methyl 2-(2,4-dichloro-5-fluoro-3-nitrobenzoyl-3-dimethylaminoacrylate of the formula X where NR⁴R⁵ represents N(CH₃)₂ & R³ represents CO₂ Me.

Into a solution of 108 gm of methyl 3-dimethylaminoethylacrylate and 92 gm of triethyl amine in toluene (1500 ml) at 70° C. -80° C. was added a solution of 2,4-dichloro-5-fluoro-3-nitrobenzoyl chloride (prepared from 200 gm of the corresponding acid and 100 gms of thionyl chloride) in 500 ml of toluene. The reaction mixture was refluxed for 6 h and cooled to room temperature. Water (1.5 lit.) was added and extracted the title product into toluene. After evaporation of solvent 300 gm of crude title product remained. A small sample can be recrystallized from methanol. MP: 123-125° C.

(ii) Preparation of methyl 2-(2,4-dichloro-5-fluoro-3-nitrobenzoyl)-3-cyclopropylaminoacrylate of the formula VIII where R represents cylcopropyl & R³ represents CO₂Me.

A solution of the crude compound prepared in step(i) in methanol (1000 ml) was treated with cyclopropylamine (54 gr) at 0-10° C. After stirring for 2-3 hrs at RT title product was filtered and washed with 200 ml of chilled methanol. to give 240.Ogr of title product. MP: 169-170° C.

(iii) Preparation of methyl 1-cyclopropyl-6-fluoro-7-chloro-8-nitro1,4-dihydro-4-oxo-3-quinolinecarboxylate of the formula XII where R represents cyclopropyl & R³ represents CO₂Me.

A mixture of 189gr of compound prepared by the process described in step(ii), 57 gr of potassium carbonate and 300 ml of DMF was heated to 60-70° C. under stirring. After maintaining for 3 hrs, the reaction mixture was cooled to 15-20° C. and filtered the solid. Filtered cake was washed with water to remove all inorganics and finally with 100 ml of methanol to give the title compound.

Yield 170 gr. MP: 226-228° C.

(iv) Preparation of methyl 1-cyclopropylfluoro-7-(1-piperazinyl)-8-nitro-1,4-dihydro-4-oxo-3-quinolinecarboxylate of the formula XIII where R represents cyclopropyl, R³ represents CO₂Me & NR¹R² represents 1-piperazinyl.

A mixture of the compound prepared in step (iii) (50 gr), DMSO (150 ml), piperazine (13 gr) and NaHCO₃ (13 gr) was stirred at RT for overnight. The reaction mixture was poured into water (400 ml) and filtered the title compound.

Yield: 55 gr. MP: 194-196° C.

(v) Preparation of methyl 1-cyclopropyl-6-fluoro-7-(4-acetyl-1 -piperazinyl)-8-nitro1,4-dihydro-4-oxo-3-quinolinecarboxylate of the formula XIII where R represents cyclopropyl R³ represents CO₂Me & NR¹ R² represents 4-acetyl-1 piperazinyl. A mixture of the compound prepared in step(iv) (33gr), acetic anhydride (70 ml) and methylenechloride (250 ml) was stirred at RT for overnight. The reaction mixture was poured into water and the title product extracted into methylenechloride and evaporation of solvent left the title compound as a light yellow solid. Yield 35gr. MP: Above 250° C. (vi) Preparation of methyl 1-cyclopropyl-6-fluoro-7-(4-acetyl-1 -piperazinyl)-8amino-1,4-dihydro4-oxo-3-quinolinecarboxylate of the formula XIV where R represents cyclopropyl, R³ represents CO₂Me, and NR¹R² represents 4-acetyl-1-piperazinyl.

A mixture of the compound prepared in step(y) (26 gr), Raney nickel (25 gr) and methanol (500 ml) were taken into an autoclave and shaken at 20-30 psi hydrogen pressure for 3-4 hrs. When the absorption of hydrogen ceased, it was filtered and the filtrate concentrated to give the title compound as a white solid.

Yield 22 gr. MP: Above 250° C.

(vii) Preparation of methyl 1-cyclopropylfluoro-7-(4-acetyl-1-piperazinyl)-1,4-dihydro-4-oxo-3-quinolinecarboxylate of the formula XV where R represents cyclopropyl, R³ represents CO₂Me, NR¹R² represents 4-acetyl-1-piperazinyl and X represents hydrogen.

A solution of the compound prepared in step(vi) (5.0 gr), and 10% H₂SO₄ (25 ml) was cooled to 0-5° C. and treated with sodium nitrite (0.96 gr) in water (3 ml). After stirring for another 15 min, the diazonium solution was added into 15% aq hypophospharous acid (50 ml) at 0-5° C. After the addition, reaction mixture was allowed to reach 25° C. and maintained overnight. The reaction mixture was poured into ice water (100 ml) and extracted the title product into methylenechloride (2×50 ml). Evaporation of solvent gave the title compound.

Yield 4.5 gr. MP: 245-247° C.

(viii) Preparation of 1-cyclopropyl-6-fluoro-7-(1-piperazinyl)-1,4-dihydro-4-oxo-3-quinolinecarboxylic acid of formula I where R represents cyclopropyl NR¹R² represents 1-piperazinyl (ciprofloxacin).

The compound prepared in step(vii) (5 gr) and aq. sodium hydroxide (2 gr in 20 ml water) was heated at 70-80° C. for 3 hrs. The solution thus obtained was cooled to RT and neutralized with acetic acid to pH 7.0. The precipitated title product was filtered and washed with water to give the title acid in quantitative yield (4.5 gr).

### EXAMPLE 2

i) Preparation of methyl 1-cyclopropyl-6-fluoro-7-(4-acetyl-1 -piperazinyl)-8chloro-1,4-dihydro4-oxo-3-quinolinecarboxylate of the formula XV where R represents cyclopropyl, R³ represents CO₂Me, NR¹R² represents 4-acetyl-1-piperazinyl, and X represents chloro group.

To a stirred suspension of methyl 1-cyclopropyl-6-fluoro-7-(4-acetylpiperazinyl)-8-amino-1,4-dihydro-4-oxo-3-quinolinecarboxylate (5 gr) and conc. HCl (25 ml) at 0° C. was added sodium nitrite (1.0 gr) in water (5 ml). After stirring for 15 min, the diazonium salt was poured into a solution of CuCl (3.0 gr) in 25ml of conc.HCl. The reaction mixture was stirred at RT for 5-6 hours and diluted with water. Title product formed was filtered and dried to yield the title compound.

Yield 5.5 gr. MP: 229-230° C.

ii) Preparation of methyl 1-cylcopropylfluoro-7-(4-acetyl-1-piperazinyl)-1,4-dihydro-4-oxo-3-quinolinecarboxylate of the formula XV where R represents cyclopropyl, R³ represents CO₂Me, NR¹R² represents 4-acetyl-1-piperazinyl and X represents hydrogen.

A solution of the chloro compound prepared in step(I) (8 gr) in methanol (200 ml) was hydrogenated with Raney nickel (4 gr) at 20-40 psi pressure of hydrogen. After completion of reaction, catalyst was filtered off and the product isolated by removal of solvent.

Yield: 7.0 gr. MP: 245-247° C.

### EXAMPLE 3

i. Preparation of methyl-1-cyclopropyl-6-fluoro-7-(4-acetyl-1-piperazinyl)-8-bromo-1,4-dihydro4-oxo-3-quinolinecarboxylate of the formula XV where R represents cyclopropyl, R³ represents CO₂Me, NR¹R² represents 4-acetyl-1-piprazinyl, and X represents bromo group.

To a stirred suspension of methyl 1-cyclopropyl-6-fluoro-7-(4-acetyl-1-piperazinyl)-8-amino-1,4-dihydro-4-oxo-3-quinolinecarboxylate (5.0 gr) and conc.HBr (25 ml) at 0-5° C. was added sodium nitrite (1.0 gr) in water (5 ml). After stirring for 15-20 min, the diazonium bromide was poured into a solution of CuBr (5.0 gr) in 25 ml of conc.HBr. The reaction mixture was stirred at 25-30° C. for 5 hrs and diluted with water. The precipitate formed was filtered and dried to yield the title compound.

Yield 5.8 gr. M.P.: 240-242° C.

ii. Preparation of methyl 1-cyclopropylfluoro-7-(4-acetyl-1-piperazinyl)-1,4-dihydro-4-oxo-3-quinolinecarboxylate of the formula XV where R represents cyclopropyl, R³ represents CO₂Me, NR¹R² represents 4-acetyl-1-piperazinyl and X represents hydrogen.

A solution of the bromocompound prepared in step(i) (5.0 gr) in methanol (150 ml) was hydrogenated with Raney nickel (2.5 gr) at 20-40 psi pressure of hydrogen. After completion of reaction, catalyst was filtered off and the product isolated by removal of solvent.

Yield:4.1 gr. M.P. 245-247° C.

### EXAMPLE 4

Preparation of methyl 1-cyclopropyl-fluoro-7-(4ethyl-1-piperazinyl)-8-nitro-1,4-dihydro-4-oxo-3-quinolinecarboxylate of the formula XIII where R represents cylclopropyl, R³ represents —CO₂Me, and NR¹R² represents 4-ethyl piperazinyl groups.

A mixture of methyl 1-cyclopropyl-6-fluoro-7-chloro-8-nitro-1,4-dihydro-4-oxo-3-quinolinecarboxylate (20 gr), DMSO (60 ml) and N-ethylpiperazine (7 gr) was stirred at RT for overnight The reaction mixture was poured into water and filtered the light yellow crystalline compound.

Yield: 24 gr. M.P: 204-206° C.

Preparation of methyl 1-cyclopropyl-6-fluoro-7-(4-ethyl-1-piperazinyl)-8-amino-1,4-dihydro-4-oxo-3-quinolinecarboxylate of the formula XIV, where R represents cyclopropyl, R³ represents —CO₂ Me, and NR¹R² represents 4-ethylpiperazinyl groups.

A mixture of the compound prepared in step(I) (20 gr), methanol (200 ml) and Raney nickel (5 gr) was stirred under hydrogen pressure (20-30 psi) for 3-4 hrs and filtered off catalyst. Removal of solvent gave the title compound.

Yield 18 gr. M.P.: 236-238° C.

iii) Preparation of methyl 1-cyclopropyl-6-fluoro-7-(ethyl-1-piperazinyl)-8-bromo-1,4-dihydro-4-oxo-3-quinolinecarboxylate of formula XV where R represents cyclopropyl, R³ represents —CO₂Me, NR¹R² represents 4-ethyl piperazinyl, and X represents bromo group.

To a stirred suspension of the compound prepared in step(ii) (10 gr) and conc.HBr (50 ml) at 0-5° C. was added sodium nitrite (2.0 gr) in 10 ml of water. After stirring for 15-20 min, the diazonium bromide solution was poured into a solution of copper (I) bromide (8.0 gr) in 40 ml of conc.HBr. The reaction mixture was stirred at RT for overnight and poured into 200 ml of water. After adjusting the pH of the reaction mass to 7.0 with solid sodium bicarbonate, title product was isolated by filtration. Yield: 11 gr. M.P.:205-206° C.

(iv) Preparation of methyl 1-cyclopropyl-6-fluoro-7-(4-ethyl-1-piperazinyl)-1,4-dihydro-4-oxo-3-quinolinecarboxylate of formula XV, where R represents cyclopropyl, R³ represents —CO₂Me, NR¹R² represents 4-ethyl pyperazinyl, and X represents hydrogen groups.

A solution of the above compound (10 gr) in methanol (100 ml) was hydrogenated with Raney nickel (3 gr) at 20-40 psi to get the title compound as light yellow solid.

Yield 9 gr. M.P.: 240-245° C.

(v) Preparation of 1-cyclopropyl-6-fluoro-7-(4-ethyl-1-piperazinyl)-1,4-dihydro-4-oxo-3-quinolinecarboxylic acid of formula I, where R represents cyclopropyl, NR¹R² represents 4-ethylpiperazinyl group.

The above compound (5 gr) was hydrolyzed using aq. sodium hydroxide (2 gr in 20 ml) at 80° C. for 3 hr and neutralized with acetic acid to get a light yellow solid.

Yield 4.5 gr. M.P: 219-221° C.

### Advantages of the Invention

1. The process utilises low temperature, which leads to higher yields(90-99%) and better purity(≧99%) of the compounds of the formula I.

2. The amount of the costly amine of the formula III (R¹R²NH) in the process requires only one equivalent.

3. The step of removing the impurity of formula VII is totally eliminated, as there is no such impurity formation in the process of the present invention.

We claim:

1. Novel compound of the formula X 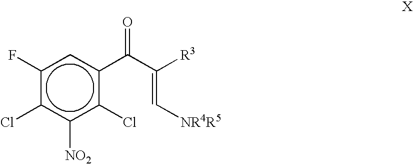
wherein NR⁴R⁵ and R³ have the meanings given above

2. Novel compound of the formula VIII 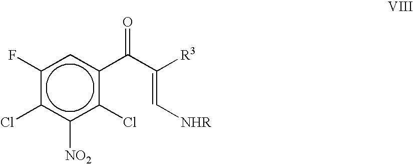
wherein R and R³ have the meanings given above.

3. Novel compound of the formula XII 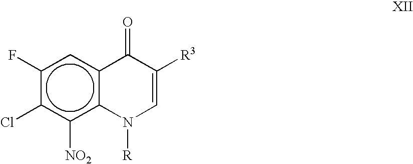
wherein R and R³ have the meanings given above.

4. Novel compound of the formula XIII 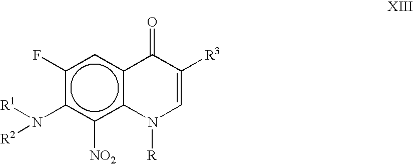
wherein R, R³ and NR¹R² have the meanings given above

5. Novel compound of the formula XIV 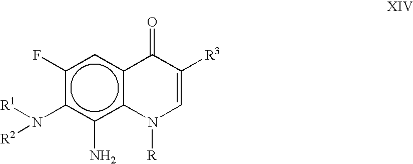
wherein R, R³ and NR¹R² have the meanings given above

6. Novel compound of the formula XV 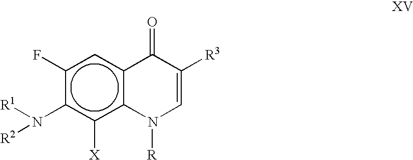
wherein R, R³,NR¹R² and X have the meanings given above

7. An improved process for the preparation of compound of the general formula I as defined above which comprises
(i) converting 2,4-dichloro-5-fluoro-acetophenone to 2,4-dichloro-5-fluoro-3-nitrobenzoic acid by conventional methods
(ii) reacting the 2,4-dichloro-5-fluoro-3-nitrobenzoyl chloride with the compound of the formula IX 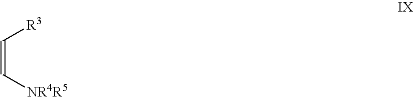
wherein R³, R⁴ and R⁵ have the meanings given above to yield the corresponding novel acrylate of the formula X 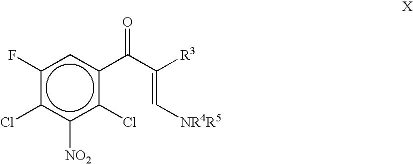
wherein NR⁴R⁵ and R³ have the meanings given above
(iii) reacting the novel compound of the formula X with a primary amine RNH₂ (of the formula XI) where R is as defined above to give a compound of the formula VIII 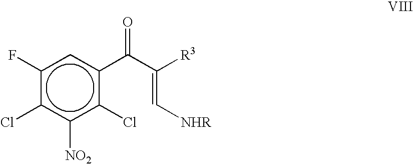
wherein R & R³ have the meanings given above
(iv) reacting the compound of the formula VIII with a base in the presence of a polar aprotic or nonpolar solvent or their mixtures to give a new compound of the formula XII 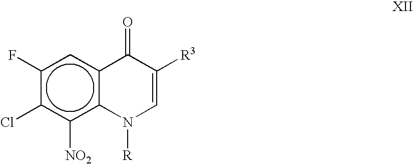
wherein R and R³ have the meanings give above
(v) reacting the novel quinolone of the formula XII with an amine R¹NHR² (of the formula III) wherein R¹ and R² have the meanings given above to give the corresponding chloro displaced novel product of the formula XIII 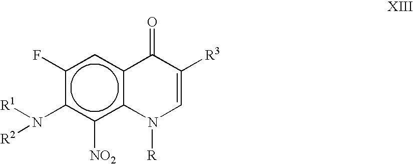
wherein R, R¹, R² & R³ have the meanings given above
(vi) subjecting the compound of the formula XIII to metal reduction by conventional methods to yield a novel compound of the formula XIV 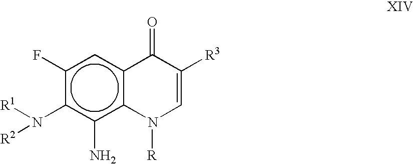
wherein R, R¹, R² & R³ have the meanings given above
(vii) deaminating the compound of the formula XIV via its diazonium salt and decomposing the salt by conventional methods to yield the compound of the formula I as defined above or decomposing the salt in the presence of copper (I) halide to give a halide derivative of the formula XV 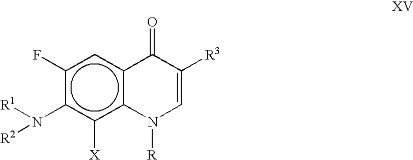
wherein X, R, R¹, R² & R³ have the meanings give above
(viii) dehalogenating the compound of the formula XV by conventional methods to yield the compound of the formula I as defined above

8. A process for the preparation of novel compound of the formula X 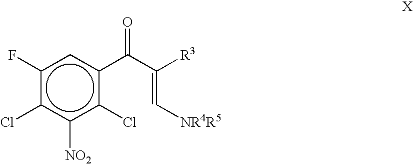
wherein R³, R⁴ and R⁵ have the meanings given above
which comprises:
(i) converting 2,4-dichloro-5-fluoro-acetophenone to 2,4-dichloro-5-fluoro-3-nitrobenzoic acid by conventional methods and
(ii) reacting the 2,4-dichloro-5-fluoro-3-nitrobenzyl chloride with the compound of the formula IX. 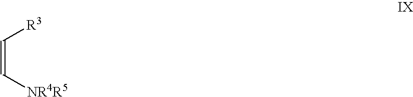
wherein R³, R⁴ and R⁵ have the meanings give above

9. A process for the preparation of novel compound of the formula XII 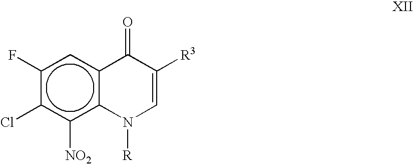
wherein R, R³ have the meanings given above.
which comprises reacting the compound of the formula VIII with a base in the presence of a polar aprotic or nonpolar solvent or their mixtures.

10. A process for the preparation of novel compound of the formula XIII which comprises reacting the novel quinolone of the formula XII with an amine R¹NHR² (of the formula III) wherein R¹ and R² have the meanings given above.

11. A process for the preparation of novel compound of the formula XIV which comprises subjecting the compound of the formula XIII to metal reduction by conventional methods.

12. A process for the preparation of novel compound of the formula XV which comprises decomposing the compound of the formula XIV in the presence of copper (I) halide to give a halide derivative of the formula XV.

13. A process as claimed in claim 1 (step 1) wherein the reaction is carried out following method contained in J. Heterocyclic Chemistry 1988, 25, 927.

14. A process as claimed in claim 1 step iii & claim 9 wherein the trans—amination is done in routine solvents such as alcohols like methanol, ethanol isopropenol etc. aromatic solvents like benzene, toluene etc. The reaction temperature being in the range of 0° C. to 60° C., the preferred temperature being in the range of 0° C. to 25° C.

15. A process as claimed in claim 1 step iv & claim 10 wherein the base employed be MOR where M represents Na, K and R represents C₁ to C₄ atoms, carbonate salts like K₂CO₃, Na₂CO₃, metal hydride like NaH.

16. A process as claimed in claim 1 step v and claim 11 wherein the polar solvent employed is selected from DMF, DMSO, DMAc etc and the nonpolar solvent can be benzene, toluene, xylene, etc.

17. A process as claimed in claim 1 step v and claim 11 wherein the reaction is effected at a temperature in the range of 25 to 100° C., preferably in the range of 25 to 40° C.

18. A process as claimed in claim 17 wherein the medium employed for the reaction is a polar aprotic solvent such as DMF, DMSO, IPA n-BuOH, t-BuOH etc. an aromatic solvent such as benzene, toluene, xyene, a halogenated solvent like CH₂Cl₂, CHCl₃, and acetonitrile.

19. A process as claimed in claim 18 wherein the reaction is conducted with one equivalent of amine and a base like Na₂CO₃, K₂CO₃, NaHCO₃, TEA, etc.

20. A process as claimed in claim 1 step vi & claim 12 wherein the reducing agent employed is selected from Raney nickel, Pd/C Fe/AcOH, Sn/HCl etc. preferably Raney nickel or Pd/C (5 to 10%).

21. A process as claimed in claim 20 wherein the medium employed is selected from alcoholic solvent such as methanol, ethanol, isopropanol etc. esters like ethyl acetate, the preferred solvent being methanol.

22. A process as claimed in claim 21 wherein the temperature employed in the reaction is in the range of 20 to 100° C., preferably 20 to 40° C., the hydrogen pressure employed being in the range of 10 to 60 psi preferably 20 to 40 psi.

23. A process as claimed in claim 1 step vii and claim 13 wherein the deamination reaction is effected via the diazonium salt derived from a compound of formula XIV which is decomposed in the presence of alcohol or hypophosphorous acid to give directly the compound of the formula I.

24. A process as claimed in claim 1 step viii and claim 14 wherein the compound of the formula XIV is decomposed in the presence of copper (I) halide to give the halo derivative of the formula XV.

25. A process as claimed in claim 1 step viii & claim 14 wherein the diazonium salt is prepared using dil H₂SO₄ medium for its direct conversion to a compound of the formula I or in dil aq HX where X represents Cl or Br medium for its conversion to a compound of the formula XV.

26. A process as claimed in claim 25 wherein the decomposition temperature employed is in the range of 5 to 80° C. preferably 15 to 30° C. for direct conversion to the compound of the formula I and in the range of 20 to 40° C. for the conversion of the halo derivative of the formula XV.

27. A process as claimed in claim 26 wherein the halo derivative of the formula XV is dehalogenated to give the compound of the formula I by treating with Raney nickel or Pd/C under hydrogen atmosphere in the presence of acid scavenger to neutralise the liberalised acid.

28. A process as claimed in claim 27 wherein the catalyst employed for the conversion is preferably 5% Pd/C, the acid scavenger employed is amines of the general formula R₃N where R represents C₁ to C₆ alkyl, or carbonate or bicarbonate salts of Na or K, preferably triethylamine.

29. A process as claimed in claims 27 & 28 wherein the temperature employed is in the range of 20 to 100° C. preferably 20 to 40° C., the hydrogen pressure employed is in the range of 10 to 60 psi preferably 20 to 40 psi, the solvent medium employed is selected from methanol, ethanol propanol, isoporpanol etc.

30. An improved process for the preparation of compound of the formula I as defined above substantially as herein described with reference to the Examples.
